(19) United States
(12) Patent Application Publication
(10) Pub. No.: US 2005/0186147 A1
(43) Pub. Date: Aug. 25, 2005

(54) Cosmetic and pharmaceutical foam with solid matter

(75) Inventors: Dov Tamarkin, Maccabim (IL); Doron Friedman, Karmei Yosef (IL); Meir Eini, Ness Ziona (IL); Alex Besonov, Rehovet (IL)

Correspondence Address: WILMER CUTLER PICKERING HALE AND DORR LLP, 60 STATE STREET, BOSTON, MA, 02109, US

(73) Assignee: Foamix Ltd.

(21) Appl. No.: 11/050,999

(22) Filed: Feb. 4, 2005

Related U.S. Application Data
(60) Provisional application No. 60/541,698, filed on Feb. 4, 2004.

(51) Int. Cl. A61L 9/04; A61K 7/42

(52) U.S. Cl. 424/47; 424/59

(57) ABSTRACT

A foamable composition includes about 2 to about 30% by weight solid particles; about 2 to about 75% by weight hydrophobic solvent; about 10 to about 85% by weight water; about 0.1% to about 5% by weight surface-active agent; about 0.1% to about 5 wt % by weight stabilizer/gelling agent; and a liquefied or compressed gas propellant in a container, which upon release provides a breakable foam suitable for topical administration.

### RELATED APPLICATIONS

This application claims priority under 35 U.S.C. §119(e) to copending U.S. Provisional Patent Application No. 60/541,698, filed Feb. 4, 2004, and entitled “Cosmetic and Pharmaceutical Foam With Solid Matter,” which is hereby incorporated in its entirety by reference.

### FIELD OF THE INVENTION

The invention relates to an alcohol-free, cosmetic or pharmaceutical foam carrier, comprising a high concentration of particulate matter and its use. More specifically, the invention relates to cosmetic or pharmaceutical foam products, comprising a high concentration of particulate matter, suitable for treatment of skin conditions. The foam products can further include water soluble and oil soluble pharmaceutical and cosmetic agents.

### BACKGROUND OF THE INVENTION

Foamable formulations are usually water based. In certain cases, such as are described in Israeli Patent Application No.152486, and Published International Application No. WO 04/037225 by the applicants of the present application, a foamable formulation may include oils, either in emulsion or as oleaginous liquid. Many drugs and cosmetic active agents are soluble in liquid vehicles, e.g., water or oil or emulsion of water and oil; other agents are insoluble in such vehicles. While foam compositions can include drugs and cosmetic active agents that are soluble in one of the composition phases (water or oil), no foam products containing a significant content of solid matter, i.e., material that is not soluble in water or oil, has been not been reported. This is probably due to the observation that solid particle matter acts as an antifoaming agent, preventing the formation of acceptable foam, and solid particles tend to sediment from the liquid composition and thus, delivery of particulate matter as part of the active composition becomes impractical.

There remains an unmet need for foam compositions comprising insoluble active ingredients useful as pharmaceuticals or cosmetics.

### BRIEF DESCRIPTION OF THE INVENTION

Despite the commonly known fact that solid particles are difficult to formulate into a foam-producing product and that such solids interfere with the foam forming ability of surfactants, we have surprisingly discovered a series of foamable carrier compositions including solid particles, which, upon admixing with a liquefied gas propellant in an aerosol container, produces a foamable composition that is suitable for topical administration. Upon discharge from an aerosol container, the composition forms a breakable foam that is rich and creamy in appearance and exhibits a very fine bubble structure. The foam does not break down immediately upon discharge; however, it collapses to spread easily onto a skin area upon slight rubbing.

In one or more embodiments of the present invention, the foamable composition includes water, a liquid non-volatile hydrophobic solvent, optionally a foam adjuvant agent selected from the group consisting of fatty acids and fatty alcohols, a surface-active agent and a water gelling agent, and at least 2% of solid particles. Such foamable compositions, when placed in an aerosol container and combined with a liquefied gas propellant, create an oil in water emulsion, which, upon release from the aerosol container, provides a therapeutically beneficial foam product. The foam retains its structure for a time sufficient for a user to apply and to rub the foam into the skin. The foam has very low yield strength and, hence, it breaks upon touch and makes rubbing easy and efficient, with an even application.

In one or more embodiments of the present invention, the foamable composition includes:
about 2% to about 30% solid particles;
about 2% to about 75% hydrophobic solvent;
about 10% to about 85% water;
about 0.1% to about 5% surface-active agent; and
stabilizer/gelling agent in a concentration sufficient to stabilize the solid in the composition, yet low enough to avoid formation of a semi-solid texture.

The foamable composition optionally further includes about 0.1% to about 5% foam adjuvant agent.

All % values are provided on a weight (w/w) basis, based on the composition without propellant (unless otherwise specified).

The cosmetic or pharmaceutical foamable carrier composition is practically a flowing liquid state, having viscosity between about 100 CPS and about 10,000 CPS, or between about 500 CPS and about 8,000 CPS or between about 1000 CPS and about 5,000 CPS.

In one or more embodiments of the present invention, the foamable composition is substantially alcohol-free, i.e., it does not contain short chain aliphatic alcohols, making it non-irritating and non-drying. Alcohols penetrate the skin's protective barrier and break down the intercellular matrix. In a recent publication by the American Academy of Dermatology (MD), titled “Facing the Facts about Skin Care Products” it is stated “[i]individuals with dry skin should avoid astringents and any product with alcohol because they easily strip away moisture from the skin” (see: www.aad.org/PressReleases FacingFacts.html). Another MD publication, titled “Sensitive About Your Skin?”, recommends to “avoid solvents that penetrate the skin including, propylene glycol and ethanol” (see: www.aad.org/PressReleases/sensitive.html).

The foamable carrier composition according to one or more embodiments of the present invention, when admixed with a propellant substance in an amount of about 5% to about 25% by weight of the total composition in an aerosol container, produces a lightweight breakable foam, suitable for facile application onto the skin, and other body areas, which may accept topically-applied products. Since the propellant in the pressurized container is in liquid state, upon admixing the foamable composition with the propellant, a stable emulsion including the oil and the propellant (jointly as the “oil phase” component of such emulsion) is formed.

The foamable compositions according to one or more embodiments of the invention optionally further include cosmetic and/or pharmaceutical agents in a therapeutically effective amount. The pharmaceutical products are useful for topical treatment of human and animal skin disorders, or any other disorder, that requires topical application of a drug. Cosmetic products are intended for beautifying the skin and improving its appearance.

The foamable composition according to one or more embodiments of the present invention provides one or more of the following advantages:
(1) The foamable composition and foamed product contains solid which is functional in treating, alleviating the symptoms of, curing or preventing a disorder of the skin, vagina, cervix, rectum and other organs responsive to topical treatment; or beautifying the appearance of the skin.
(2) The foam enables even and uniform spreading of the solid matter over the target area.
(3) The foam is lightweight and thus, economical.
(4) The foam contains a hydrophobic solvent, in any desirable concentration, which provides refatting, protective and skin soothing effect.
(5) The foam can further include pharmaceutical and cosmetic active agents, both water soluble and oil soluble.
(6) The foam is easily spreadable, allowing treatment of large areas such as the arms, back, legs and the breast.
(7) Due to its flow properties, the foam spreads effectively into folds and wrinkles, providing uniform distribution of the active agent without the need of extensive rubbing and absorbs into the skin.

### DESCRIPTION OF THE DRAWINGS

A more complete appreciation of the present invention and many of its advantages will be understood by reference to the following detailed description when considered in connection with the following drawings, which are presented for the purpose of illustration only and are not intended to limit the scope of the appended claims, and in which:

FIG. 1 illustrates the uniform dispersion of zinc oxide 10% in the foam, following discharge from the pressurized can; and

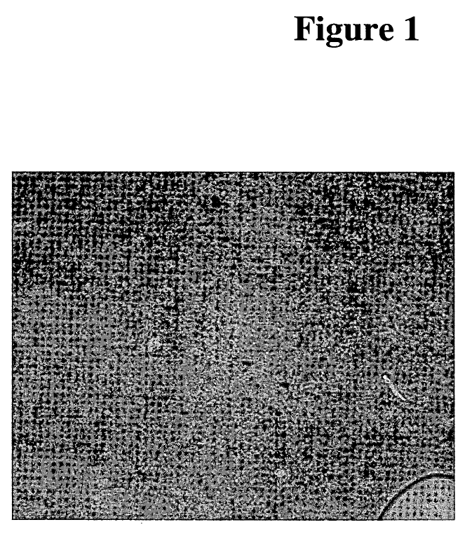

FIG. 2 illustrates the masking effect of titanium dioxide 2% foam on hyperpigmented skin, illustrating the even distribution on the skin surface, thereby providing effective sun protection and immediate whitening effect.

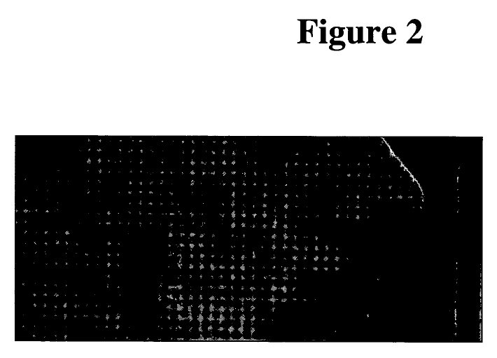

### DETAILED DESCRIPTION OF THE INVENTION

For convenience certain terms employed in the specification, examples and claims are described herein.

According to the present invention, solid matter or particulate matter shall mean material that is not soluble in the liquid carrier composition of the foamable composition. For definition purposes, solid matter shall mean material that is not soluble in the foamable composition more than 10% of the concentration intended to be included in the foamable composition. The concentration of the solid matter in the foamable composition is from about 2% to about 40% w/w. In one or more embodiments, the concentration of solid matter in the composition is from about 5% to about 40% w/w. In one or more embodiments, the concentration of solid matter in the composition is from about 10% to about 25% w/w.

By way of example, the following classes of solid matter substances are presented:
Metallic oxides, such as titanium dioxide, zinc oxide, zirconium oxide, iron oxide. Preferably, as used in the present invention, titanium dioxide has an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. In one embodiment the metal oxides are present in the amount of from about 0.1% to about 20%, preferably from about 0.5% to about 16%, more preferably from about 1% to about 10%, of the composition; In yet another embodiment, such solids are micronized to form particles having primary size of less than 15 nm.
Silicon containing solid matter includes silicone oxide, also termed “silica” and silica gel”, a white or colorless vitreous insoluble solid (SiO2); and talc, which is fine grained mineral consisting of hydrated magnesium silicate;
Carbon, for example in the form of amorphous carbon or graphite;
Oxidizing agents, such as benzoyl peroxide, calcium and magnesium hypochlorite;
Metallic Silver, in small particles, including nanocrystalline silver, which is used for antibacterial and wound healing purposes;
Other metal particles and mineral particles;
Cosmetic scrub materials, including, for example meals of strawberry seeds, raspberry seeds, apricot seeds, sweet almond, cranberry seeds;
Pigments, which are insoluble in the foamable composition.

A hydrophobic solvent according to the present invention is a liquid material having solubility in distilled water at ambient temperature of less than about 1 gm per 100 mL, or less than about 0.5 gm per 100 mL, or less than about 0.1 gm per 100 mL. It is liquid at ambient temperature.

The total content of hydrophobic solvent may vary from about 2% to about 75% (w/w) of the foamable composition. Generally, higher hydrophobic solvent concentrations are more appropriate for the treatment of dry skin, and/or for the treatment of a disease, which is more responsive to drugs delivered in an oily vehicle. Likewise, the higher oil-content composition classes provide an enhanced occlusive effect, which in turn induces the skin penetration of an active agent. Another consideration relates to user acceptance of a product containing a high concentration of the hydrophobic solvent (from about 25% of the composition), which would leave some oily feeling post-application. Thus, a particular composition of the present invention is selected having a hydrophobic solvent concentration in view of the target population and its specific needs.

In one or more embodiments of the present invention, the hydrophobic solvent is mineral oil. Mineral oil (Chemical Abstracts Service Registry number 8012-95-1) is a mixture of aliphatic, naphthalenic, and aromatic liquid hydrocarbons that are derived from petroleum. It is typically liquid; its viscosity is in the range of about 35 CST to about 100 CST (at 40° C.), and its pour point (the lowest temperature at which an oil can be handled without excessive amounts of wax crystals forming) is below 0° C.

Yet other hydrophobic solvents include liquid oils from vegetable, marine or animal sources. By way of example, the unsaturated oil may be selected from the group consisting of olive, corn, soybean, canola, cottonseed, coconut, sesame, sunflower, borage seed, syzigium aromaticum, hempseed, herring, cod-liver, salmon, flaxseed, wheat germ and evening primrose oils and mixtures thereof, at any proportion.

Yet another class of oils includes polyunsaturated oils, e.g., esters, and in particular glyceryl esters, of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Thus, in one or more embodiments of the present invention the hydrophobic solvent includes at least 6% by weight foamable composition of an oil selected from omega-3 oil, omega-6 oil, and mixtures thereof.

Another class of oils suitable for use as a hydrophobic solvent is liquid hydrophobic plant-derived oils, or essential oils, e.g. “therapeutic oils” containing active biologically occurring molecules that have a therapeutic effect when applied topically. Examples of such oils include rosehip oil, which contain retinoids and is known to reduce acne and post-acne scars, and tea tree oil, which possess antibacterial, antifungal and antiviral properties. Other examples of essential oils are oils of basil, camphor, cardamom, carrot, citronella, clary sage, clove, cypress, frankincense, ginger, grapefruit, hyssop, jasmine, lavender, lemon, mandarin, marjoram, myrrh, neroli, nutmeg, petitgrain, sage, tangerine, vanilla, verbena, as well as any other therapeutically beneficial oil, know in the art of herbal medication.

In one or more embodiments of the present invention, the hydrophobic solvent is an “emollient”. An emollient is a hydrophobic agent that softens, smoothens and improves lipid content of the skin or other mucous membranes. In one or more embodiments of the present invention, the emollient is a liquid. Without derogating the generality of this definition, examples of suitable emollients for use include isostearic acid derivatives, isopropyl palmitate, lanolin oil, diisopropyl dimerate, diisopropyl adipate, dimethyl isosorbide, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, octyl dodecanol, octyl hydroxystearate and mixtures thereof. Other examples of other suitable emollients can also be found in the Cosmetic Bench Reference, pp. 1.19-1.22 (1996). In one or more embodiments, the hydrophobic solvent is a mixture of a mineral oil or silicone oil and an emollient.

In one or more embodiments of the present invention, silicone oil is a component of the hydrophobic solvent. Silicone oils are useful in foamable compositions due to their known skin protective and occlusive properties. Suitable silicone oils for use in the invention include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are preferably chosen from cyclic or linear polydimethylsiloxanes containing from about 3 to about 9, preferably from about 4 to about 5, silicon atoms. Volatile silicones such as cyclomethicones can also be used. Water-soluble silicones, such as dimethicone copolyol are not included in the definition of silicone oils (as hydrophobic solvents) according to the present invention.

The hydrophobic solvent of the present invention may include a mixture of two or more of the above hydrophobic solvents in any proportion.

Gelling/stabilizing agents according to one or more embodiments of the present invention stabilize the aqueous phase by, for example, increasing viscosity and linking capability. Exemplary gelling/stabilizing agents that can be used in accordance with one or more embodiments of the present invention include for example, but are not limited to, naturally-occurring polymeric materials such as, locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling/stabilizing agents include the acrylic acid/ethyl acrylate copolymers and the carboxyvinyl polymers sold, for example, by the B.F. Goodrich Company under the trademark of Carbopol Registered TM resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1% of a polyallyl ether of sucrose having an average of about 5.8 allyl groups for each sucrose molecule.

In an embodiment, the gelling/stabilizing agent is a cellulose polymer.

In a further embodiment, the gelling/stabilizing agent is microcrystalline cellulose. Microcrystalline cellulose is basically cellulose and is derived from high quality wood pulp. While cellulose is the most abundant organic material, microcrystalline cellulose can only be derived from a special grade of alpha cellulose. A naturally occurring polymer, it is included of glucose units connected by a 1-4 beta glycosidic bond. These linear cellulose chains are bundled together as microfibril spiralled together in the walls of plant cell. Each microfibril exhibits a high degree of three-dimensional internal bonding resulting in a crystalline structure that is insoluble in water and resistant to reagents. There are, however, relatively weak segments of the microfibril with weaker internal bonding. These are called amorphous regions but are more accurately called dislocations since microfibril containing single-phase structure. The crystalline region is isolated to produce Microcrystalline Cellulose.

Yet, in an additional embodiment, the gelling/stabilizing agent is a combination of microcrystalline cellulose and a second gelling agent, selected from carboxymethyl cellulose, carboxyethyl cellulose or carboxypropyl cellulose and salts and derivatives thereof.

The concentration of the gelling/stabilizing agent is sufficient to stabilize the solid in the composition, yet, low enough to avoid formation of semi-solid texture. Suitable gelling/stabilizing agent concentration should be adjusted, to create viscosity between about 100 CPS and about 10,000 CPS, more preferably between about 500 CPS and about 8,000 CPS and most preferably between about 1000 CPS and about 5,000 CPS.

Thus, according to one or more embodiments of the present invention, the gelling/stabilizing agent is present in a concentration in the range of about 0.1% to about 5% (wt) of the foamable composition. The concentration is in the range of about 0.5% to about 3 wt % or the concentration is in the range of about 1% to about 2 wt % of the foamable composition. In one or more embodiments, it is typically less than 1 wt % of the foamable composition.

In an embodiment, the gelling agent or agents denote thixotropic properties to the composition, a semi-solid gel state at rest and liquid or viscous liquid under shear. Semi-solid properties at rest contribute to increased physical stability and stabilization of the solid particulate matter, whereas liquefying under shear enables flow of composition through the closures orifice and production of foam.

It has been unexpectedly found that it is possible to stabilize large amount of non-dissolving particulate matter with very minimal sedimentation, while maintaining enough flow properties to produce foam from the composition packed under pressure with the propellants.

Manual hand shaking or agitating of the composition provides sufficient shear stress on the thixotropic composition, that break the gel structure and allow from propagation.

Hence in one or more preferred embodiments of the present invention, the gelling agent or agent is hydrocolloid, selected from the group of natural cellulose gums and salts and derivatives thereof, polysaccharides and salts and derivatives thereof, microcrystalline cellulose, sodium carboxymethyl cellulose, fumed silica, bentonite, xanthan gum, carrageennan, polyacrylate and mixtures thereof.

Surface-active agents, according to the present invention include any agent linking oil and water in the composition and stabilizing oil in water or water in oil compositions.

The surface-active agent is suitably selected from anionic, cationic, nonionic, zwitterionic, amphoteric and ampholytic surfactants, as well as mixtures of these surfactants. Such surfactants are well known to those skilled in the pharmaceutical and cosmetic formulation art. Nonlimiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

A combination of surface active agents is possible. Any surface-active agent or combinations thereof may be used as surface-active agent. According to one or more embodiments of the present invention, the surface-active agent (or agents) has an HLB of higher than 8 and more preferable higher than 12.

Optionally, foam adjuvants are included in the foamable compositions of the present invention to increase the foaming capacity of surfactants and/or to stabilize the foam. In one or more embodiments of the present invention, the foam adjuvant agents includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1 -triacontanol (C30), as well as alcohols with longer carbon chains (up to C50). Fatty alcohols, derived from beeswax, including a mixture of alcohols, a majority of which has at least 20 carbon atoms in their carbon chain, are especially well suited as foam adjuvant agents according to the present invention. The concentration of the fatty alcohol, required to support the foam system is inversely related to the length of its carbon chains.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof.

Optionally, the carbon atom chain of the fatty alcohol or the fatty acid may have at least one double bond. A further class of foam adjuvant agent according to the present invention includes a long chain fatty alcohol or fatty acid, wherein the carbon atom chain is branched. The carbon chain of the fatty acid or fatty alcohol can be substituted with a hydroxyl group, such as 12-hydroxy stearic acid.

The foam adjuvant agent according to one or more embodiments of the present invention includes a mixture of fatty alcohols, fatty acids and hydroxy fatty acids and derivatives thereof in any proportion, providing that the total amount is about 0.1% to about 5% (w/w) of the carrier mass. More preferably, the total amount is about 0.4% to about 2.5% (w/w) of the carrier mass.

While fatty alcohols and fatty acids serve to stabilize the resultant foam composition, they often provide additional therapeutic properties. Long chain saturated and mono unsaturated fatty alcohols, e.g., stearyl alcohol, erycyl alcohol, arachidyl alcohol and docosanol have been reported to possess antiviral, anti infective, anti-proliferative and anti-inflammatory properties (U.S. Pat. No. 4,874,794). Longer chain fatty alcohols, e.g., tetracosanol, hexacosanol, heptacosanol, octacosanol, triacontanol, etc. are also known for their metabolism modifying properties and tissue energizing properties. Long chain fatty acids have also been reported to possess anti-infective characteristics. Thus, the pharmaceutical or cosmetic carrier, containing the foam adjuvant agent of the present invention provides an extra therapeutic benefit in comparison with currently used vehicles, which are inert and non-active.

In a recent publication by the American Academy of Dermatology (AAD), titled “Facing the Facts about Skin Care Products” it is stated “[i]ndividuals with dry skin should avoid astringents and any product with alcohol because they easily strip away moisture from the skin” (see: www.aad.org/PressReleases FacingFacts.html). Another AAD publication, titled “Sensitive About Your Skin?”, recommends to “avoid solvents that penetrate the skin including, propylene glycol and ethanol” (see: www.aad.org/PressReleases/sensitive.html).

In one or more embodiments of the present invention, the foamable carrier composition is substantially alcohol-free, i.e., it does not contain short chain aliphatic alcohols, making it non-irritating and non-drying. Alcohols penetrate the skin's protective barrier and break down the intercellular matrix. The term “substantially alcohol-free” as used herein refers to a concentration of about 5% or less alcohol.

In many cases, the inclusion of an additional therapeutic agent in the foamable pharmaceutical of the present invention, contributes to the clinical activity of the composition. For example, it is known that keratolytic agents, such as alpha hydroxyl acids, beta hydroxyl acids, retinoids, etc., contribute to the clinical efficacy of an antifungal agent. Likewise, it is known, for example, that the addition of a second anti-infective agent, such as an antibacterial agent and antiviral agent, an anti-parasite agent or a second antifungal agent is beneficial in the treatment of a complex infectious condition. An additional non-limiting example is of an additional therapeutic agent is an anti-inflammatory agent, which contributes to therapy by treating the inflammatory reaction, which accompanies many infective conditions.

Thus, in one or more embodiments, the foamable composition further includes at least one additional therapeutic agent, in a therapeutically effective concentration.

In one or more embodiments, the at least one additional therapeutic agent is selected from the group consisting of an anti-infective, an antibiotic, an antibacterial agent, an antifungal agent, an antiviral agent, an antiparasitic agent, an antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an analgesic, an antiallergic agent, a corticosteroid, a non-steroidal anti-inflammatory drug, an alpha hydroxyl acid, a beta-hydroxy acid, a protein, a peptide, a neuropeptide, a allergen, an immunogenic substance, a haptene, an oxidizing agent, an antioxidant, a retinoid, an antiproliferative agent, an anticancer agent, a photodynamic therapy agent, an anti-wrinkle agent, a radical scavenger, a self-tanning agent, a skin whitening agent, a skin protective agent, an anti-cellulite agent, a massaging oil and an anti-wart agent, a refatting agent, a lubricating agent and mixtures thereof.

The pharmaceutical or cosmetic foam carrier of the present invention may further optionally include a variety of pharmaceutical or cosmetic ingredients, which are added in order to fine-tune the consistency of the formulation, protect the formulation components from degradation and oxidation and bestow their cosmetic acceptability. Such excipients may be selected, for example, from the group consisting of diglycerides, triglycerides, stabilizing agents, antioxidants, humectants, flavoring, colorant and odorant agents and other formulation components, used in the art of pharmaceutical and cosmetic formulary.

A pharmaceutical or cosmetic composition manufactured using the foamable composition according to the present invention is very easy to use. When applied onto the afflicted body surface of humans or animals, it is in a foam state, allowing free application without spillage. Upon further application of a mechanical force, e.g., by rubbing the composition onto the body surface, it freely spreads on the surface and is rapidly absorbed.

Aerosol propellants are used to generate and administer the foamable composition as foam. The total composition including propellant, foamable compositions and optional ingredients is referred to as the foamable carrier. The propellant makes up about 5 to about 25 wt % of the foamable carrier. Examples of suitable propellants include volatile hydrocarbons such as butane, propane, isobutane or mixtures thereof, and fluorocarbon gases.

The composition including water, hydrophobic solvents, formulation excipients and propellant, may be formed as a stable emulsion having an acceptable shelf-life.

The composition is free flowing, since otherwise it cannot flow through the dip-tube of the aerosol container and create acceptable foam. Compositions including semi-solid hydrophobic solvents, e.g., white petrolatum, are excessively viscous and demonstrate poor flowability.

The combination of a surface active agent, foaming adjuvant and water gelling agent according to one or more embodiments of the invention provides a low specific gravity foam having superior flow properties and sheer breakability (among other attributes). According to one or more embodiments of the present invention, the total amount of surface active agent, foaming adjuvant and water gelling agent, in combination does not exceed about 8% (w/w) of foamable composition. In other embodiments, the combined amounts of surface active agent, foaming adjuvant and water gelling agent is less than 5% (w/w) of foamable composition. The low solids content improves the flow properties of the foam, reduces unpleasant skin residue and reduces the cost of manufacture. As is demonstrated herein, the foam quality and foam breakability is excellent, despite the low levels of these components in the foam.

The following scale for foam quality is used to evaluate foams:
E (excellent): very rich and creamy in appearance, does not show any bubble structure or shows a very fine (small) bubble structure.
G (good): rich and creamy in appearance, very small bubble size, “dulls” more rapidly than an excellent foam.
FG (fairly good): a moderate amount of creaminess noticeable, bubble structure is noticeable.
F (fair): very little creaminess noticeable, larger bubble structure than a “fairly good” foam.
P (poor): no creaminess noticeable, large bubble structure.
VP (very poor): dry foam, large very dull bubbles, difficult to spread on the skin.

Foams that are adequate for topical administration are typically of quality grade E or G, upon release from the aerosol container. Smaller bubbles mean more stable foam, which does not collapse spontaneously immediately upon discharge from the container. The finer foam structure looks and feels smoother, thus increasing its usability and appeal.

A feature of a foamable composition is that the solid particles do not easily precipitate out or sediment from the composition. In order to distinguish between acceptable and unacceptable compositions in terms of sedimentation, we have used the centrifugation test, by subjecting the composition to centrifugation at 10000 RPM. This was done first for 3 minutes, followed by a 10-minutes test. Compositions that showed significant sedimentation after 3 minutes at 10000 RPM were rejected.

A further foam property is breakability. Sheer-force breakability of the foam is clearly advantageous over the thermally-induced breakability that is found, for example, in U.S. Pat. No. 6,126,920, and the respective Olux™ and Luxiq™ products. According to the use instructions of Olux™ and Luxiq™, these foams cannot be applied on the hand and afterwards delivered to the afflicted area, since it immediately collapses upon exposure to skin temperature.

Yet, another property of a foamable composition is specific gravity of the foam, as measured upon release from the aerosol can. Typically, foams have specific gravity of less than about 0.1 g/mL and preferably, less than about 0.05 g/mL.

There are many applications for a foam that includes solid matter. Below is a non-limiting list of applications in the healthcare area, which are provided to demonstrate the versatility of such a composition. While many of such applications are in the healthcare area, solid-containing foams can be used in many other applications, including for example mechanics, electronics and sanitation.

Generally, products for the prevention and treatment of diaper dermatitis are provided in the form of paste that is intended for application on the baby's posterior, under the diaper. The paste usually includes about 30% oil and/or petrolatum, and about 10% zinc oxide, which are intended to provide a protective barrier between the baby's skin and the irritating environment inside the diaper. While containing the right ingredients, current baby pastes are very viscous and thick, and therefore hard to spread on the target area.

The foam for diaper rash of the present invention includes the following ingredients:
about 6% to about 20% zinc oxide (or an alternative metal oxide)
about 10% to about 40% hydrophobic solvent;
about 40% to about 80% water
about 0.1% to about 5% surface-active agent; and
about 0.5% to about 5% stabilizer/gelling agent, preferably from about 1% to about 2% stabilizer/gelling agent
optionally, about 0.1% to about 5% foam adjuvant agent;

Such foam is superior to current pastes in that it is very fluffy and light. Upon discharge from the aerosol can, it creates a mass, having density between 0.01 gr/mL and 0.1 gr/mL, which is very easy to spread evenly and uniformly on the target area. There is no need to rub thoroughly and therefore, application of the foam does not cause any discomfort to the baby, unlike conventional baby pastes. Furthermore, the application is much more comfortable to the one who applies the foam and thus, treatment compliance is enhanced.

Medicated foams for diaper dermatitis may further include anti-irritating and anti-infective agents, as exemplified below:
a. Corticosteroids, anti-inflammatory, anti-irritant and anti-allergic agents. For irritated diaper rash, corticosteroid drugs, such as hydrocortisone, as well as non-steroidal anti-inflammatory agents, anti-irritant agents and antiallergics agents, in a therapeutically effective concentration can be added to the foam. The resulting foam helps decrease the inflammation. Further examples of suitable corticosteroids, non-steroidal anti-inflammatory agents, anti-irritant agents and antiallergic agents are provided below.
b. Anti-fungal agents. For the treatment of rashes in which fungal and/or yeast infection, antifungal drugs, including chemically derived or plant derived substances, in a therapeutically effective concentration, can be included in the foam. Such drugs can be chemically derived or extracted from herbal substances. Examples of suitable anti-fungal agents, classified by chemical families, are provided below.
c. Anti-microbial agents. Various anti-microbial agents, including chemically derived or plant derived substances, in a therapeutically effective concentration, can also be included in the foam, in order to provide effective protection against bacterial infection. Examples of suitable anti-microbial agents, classified by chemical families, are provided below.

An example of a skin protective foam according to one or more embodiments of the present invention includes the following ingredients:
about 6% to about 20% metal oxide of mineral solid matter
about 10% to about 40% hydrophobic solvent;
about 40% to about 80% water
about 0.1% to about 5% surface-active agent; and
about 0.5% to about 5% stabilizer/gelling agent, more preferably from about 1% to about 2% stabilizer/gelling agent
optionally, about 0.1% to about 5% foam adjuvant agent;

In addition to the above components, further therapeutic agents selected from the group of anti-irritants, corticosteroids, antibacterial agents and anti-fungal agents in a therapeutically effective concentration can be incorporated in the foam.

In one or more embodiments according to the present invention, the solid matter has anti-infective properties. Silver particles, mainly in their colloidal form, are known to exert anti-bacterial, anti-fungal and anti-viral effects, when applied topically on an afflicted area. Due to these properties, silver can be used to fight existing infections and protect from new infestation. Furthermore, silver can be used for protection of human and animal subjects and curing the risk of chemical and biological warfare. Silver is also known to induce wound and burn healing. Thus, a foam including silver particles and colloidal silver in a therapeutically effective concentration has clear benefits for the treatment of such conditions.

Another exemplary solid antibacterial agent is benzoyl peroxide (BPO), which is used for example in the treatment of acne. In order to be effective, BPO should be administered in a composition including at least 5% BPO and preferably 10% BPO. Inclusion of 5% and 10% BPO in the foam of the present invention provides a more convenient way to treat acne and other disorders which respond to topical administration of BPO.

The foamable composition is particularly suitable for the uniform delivery of a skin lightening agent. In one or more embodiments of the present invention, the foam composition includes a combination of a skin whitening agent and an inorganic metal oxide solid matter. When inorganic metal oxide agents, e.g. titanium dioxide and zinc oxide are rubbed onto the skin, they leave a white coating, which provides an instant (although transient) whitening effect, which is highly desirable by the consumer, who wishes to see instant change in his/her appearance. The whitening agent, in combination with the inorganic sunscreen agent in the foam carrier can be easily and uniformly distributed on the skin surface, thereby affording an instant even and uniform whitening effect, unlike creams that are difficult to spread evenly on skin areas.

The composition may contain from about 0.1% to about 10%, or from about 0.2% to about 5%, of the composition, of a skin-lightening agent. Suitable skin lightening or whitening agents include those known in the art, including hydroquinone, azelaic acid and other related di-carboxylic acids, and salts and derivatives thereof, retinoids, kojic acid, arbutin, nicotinic acid and its precursors, salts and derivatives, ascorbic acid and salts and derivatives thereof (e.g., magnesium ascorbyl phosphate or sodium ascorbyl phosphate), and herbal extracts (e.g., licorice extract, mulberry extract, placental extract).

Exposure to ultraviolet light can result in excessive scaling and texture changes of the stratum corneum. The foam of the present invention is advantageous for the delivery of sunscreen agents. Its application is very convenient and it spreads easily over large skin areas.

Inorganic solid sunscreens are very useful in blocking both UVA and UVB radiation. Such solid sunscreen herein may include, by way of example, the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, of the composition.

A wide variety of conventional organic sunscreen active agents can further be included in the composition, in order to attain higher SPF values, including, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

In one or more embodiments, solid matter incorporated into the composition may be scrub materials, including silica gel, and botanical scrub materials, for example meals of strawberry seeds, raspberry seeds, apricot seeds, sweet almond and cranberry seeds. Anti-microbial and other anti-infective agents can be added.

In one or more embodiments, the solid matter includes insoluble pigments in the foamable composition in a concentration suitable for coloring the skin.

As discussed in the description above, the foamable composition may also include soluble pharmaceutical and cosmetic active agents (collectively, “active agents”). Such active agents may consist of a single agent or a combination of agents that can be dissolved in the water phase or the hydrophobic phase of the carrier composition. Examples of such drugs are antibiotic, antibacterial, antifungal, antiviral, antiinflammatory, anesthetic, analgesic, antiallergic, corticosteroid, retinoid and antiproliferative medications and mixtures thereof at any proportion. The concentration of drugs may be adopted to exert a therapeutic effect on a disease when applied to an afflicted area. Below, some of these agents are detailed:

By way of example, the antibacterial drugs can be selected from the group of chloramphenicol, tetracyclines, synthetic and semi-synthesic penicillins, beta-lactames, quinolones, fluoroquinolnes, macrolide antibiotics, metronidaziole and its derivatives and analogs, dicarboxylic acids, such as azelaic acid, slicylates, peptide antibiotics, cyclosporines and any combination thereof at a therapeutically effective concentration. Another group of antibacterial agents which is non-specific, includes strong oxidants and free radical liberating compounds, such as hydrogen peroxide, bleaching agents (e.g., sodium, calcium or magnesium hypochloride and the like) iodine, chlorohexidine and benzoyl peroxide. An exemplary list of anti-microbial and anti-fungal plant extracts and essential oils is provided in K A Hammer, C F Carson and T V Riley, “Antimicrobial activity of essential oils and other plant extracts”, J. Applied Microbiology 86 (1999) 985-990.

The composition may further include an anti-fungal drug, which is active against dermatophytes and candida, selected from the group of, but not limited to azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration. Other anti-fungal agents can be selected form the groups of herbal extracts and essential oils, which are known by those skilled in the art of natural therapy to possess ant-fungal properties. An exemplary list of anti-microbial and anti-fungal plant extracts and essential oils is provided in K A Hammer, C F Carson and T V Riley, “Antimicrobial activity of essential oils and other plant extracts”, J. Applied Microbiology 86 (1999) 985-990.

The composition may further include anti-viral agents selected from any known antiviral agents, including, in a non-limiting fashion, Vidarabine; Acyclovir; Gancyclovir; Nucleoside-analog reverse transcriptase inhibitors (NRTI), e.g., AZT (zidovudine), ddl (didanosine), ddC (zalcitabine), d4T (stavudine), 3TC (lamivudine); non-nucleoside reverse transcriptase inhibitors (NNRTI), e.g., nevirapine, delavirdine; protease inhibitors, such as saquinavir, ritonavir, indinavir, nelfinavir, ribavirin, amantadine/aimantadine; and interferons.

The composition may further include antiinflammatory or antiallergic agent selected from the group of corticosteroids, non-steroidal antiinflammatory drugs (NSAIDs), anti-histamines, immunosuppressants and any combination thereof at a therapeutically effective concentration. The following table provides a summary of 10 currently available corticosteroid agent and their typical therapeutically effective concentration.

| Potency | Compound |
|---|---|
| Very high | Clobetasol proprionate |
| | Halobetasol proprionate |
| High | Betamethasone diproprionate |
| | Betamethasone valerate |
| | Fluocinolone acetonide |
| | Halcinonide |
| Medium | Betamethasone valerate |
| | Fluocinolone acetonide |
| | Hydrocortisone valerate |
| | Triamcinolone acetonide |
| Low | Hydrocortisone |

A second class of anti-inflammatory agents, which is useful in the foam of the present invention, includes the nonsteroidal anti-inflammatory agents (NSAIDs). The 15 variety of compounds encompassed by this group is well-known to those skilled in the art. Specific non-steroidal anti-inflammatory agents useful in the composition invention include, but are not limited to:
1) Oxicams, such as piroxicam, isoxicam, tenoxicam, sudoxicam;
2) Salicylates, such as salicylic acid, ethyl salicylate, methyl salycilate, aspirin, disalcid, benorylate, trilisate, safapryn, solprin, diflunisal, and fendosal;
3) Acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
4) Fenamates, such as mefenamic, meclofenamic, flufenamic, niflumic, and tolfenamic acids;
5) Propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
6) Pyrazoles, such as phenylbutazone, oxyphenbutazone, feprazone, azapropazone, and trimethazone.

Any further steroidal and nonsteroidal compounds having the capacity to prevent, alleviate the symptoms of, treat or cure inflammation processes, are generally included as possible anti-inflammatory agents.

Topical antihistaminic preparations currently available include 1% and 2% diphenhydramine (Benadryl® and Caladryl®), 5% doxepin (Zonalon®) cream, phrilamine maleate, chlorpheniramine and tripelennamine, phenothiazines, promethazine hydrochloride (Phenergan®) and dimethindene maleate. These drugs, as well as additional antihistamins can also be incorporated in the composition of the present invention.

Examples of local anesthetic agents include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof. Mixtures of such anesthetic agents may be synergistically beneficial.

The term “keratolytically active agent” is used herein to mean a compound that loosens and removes the stratum corneum of the skin, or alters the structure of the keratin layers of skin. Keratolytically active agents are used in the treatment of many dermatological disorders, which involve dry skin, hyperkeratiinization (such as prsoriasis), skin itching (such as xerosis), acne and rosacea.

Suitable keratolytically active agent include phenol and substituted phenolic compounds. Such compounds are known to dissolve and loosen the intracellular matrix of the hyperkeratinized tissue. As such, they are used in the treatment of dermatological disorders. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic. These compounds also exhibit antiseptic properties. Cresols also possess bactericidal and keratolytic properties.

Vitamin A and its derivatives, such as retinoic acid, isoretinoic acid, retinol and retinal are another preferred class of keratolytically active agents.

Another group of keratolytically active agents include alpha-hydroxy acids, such as lactic acid and glycolic acid and their respective salts and derivatives; and beta-hydroxy acids, such as Salicylic acid (o-hydroxybenzoic acid) and its salts and pharmaceutically acceptable derivatives, which typically possess anti-inflammatory, as well as keratolytic, activity.

Yet, another class of preferred keratolytically active agents includes urea and its derivatives.

Examples of acceptable retinoids are etretinate, actiretin, isotretinoin, adapalene and tazarotene are further examples of the retinoid isomers and analogs.

There are several types of insect repellents to use when protecting people and animals from flying or biting insects, spiders, ticks and mites. By way of example, these may include DEET (N,N-diethyl-m-toluamide), dimethyl phthalate, piperonyl butoxide and permethrin. Insect repelling terpenoids, have been reported by Hwang, et al, J. Chem. Ecol., 11, 1297 (1985); and Ruledge, J. Am. Mosquito Control Assoc. 4, 414 (1988).

A particularly preferred group of insect repellents includes the terpenoid compounds, described in U.S. Pat. No. 5,411,992, including:
(1) Terpenoid-alcohol or terpene-ols are terpenoids which have at least one hydroxyl group. Examples of terpene-ols include: C10H16O compounds, perillyl alcohol, carveol, myrtenol, and cis-verbenol; C10H18O compounds, myrtanol, iso-pinocampheol, dihydrocarveol, isopulegol, terpineol, terpinen-4-ol, nerol, geraniol, and linalool, and C10H20O compounds, menthol, beta-citronellol, and dihydro-myrcenol.
(2) Terpenoid-esters are terpenoids, which have at least one ester group which is the product of the bonding of the hydroxyl group of a terpene-ol with an aliphatic carboxylic acid that can contain functional groups such as the hydroxyl or amine on the aliphatic chain. Examples of suitable aliphatic carboxylic acids include acetic acid, propionic acid, lactic acid, and various amino acids. Examples of terpenoid-esters include: carvyl acetate, carvyl propionate, and menthyl lactate.
(3) Essential oils which contain terpenoids and perfumes which contain terpenoids. Non-limiting examples of essential oils which have high content of terpene-ols and esters include bergamot (62% terpenoids); sage (>50% terpenoids); styrax (>50% terpenoids); peppermint (>50% terpenoids); and pine Siberian (75% terpenoids %). Terpenes, aldehydes and ketones vary in their usefulness but as a general group have potential as insect-repellent.

The foamable composition is particularly suitable for the effective uniform spreading of a protective layer, including sold matter and an insect repellent agent onto large areas of the skin of humans and animals. The hydrophobic solvent present in the foam composition helps retain the insect repellent on the skin surface for an extended period of time.

Yet, in a further embodiment, the foamable composition is suitable for delivery of insect-killing agents (insecticides) to an afflicted external surface area of humans and animals. Thus, the pharmaceutical or cosmetic composition may include an insecticide, known in the art of parasitology. By way of example, such insecticide can be selected selected from the group of permethrin, hexachlorobenzene, carbamate, naturally occuring pyrethroids, permethrin, allethrin, malathion, piperonyl butoxide and any combination thereof at a therapeutically effective concentration. Its application is very convenient and it spreads easily, even over hairy areas. The hydrophobic solvent present in the foam composition helps retain the insecticide on the treated area for an extended period of time. Furthermore, the presence of a hydrophobic solvent in the foam eases mechanical removal of lice and nits with a comb.

In addition to the solid matter, the compositions may include a safe and effective amount of one or more soluble anti-acne active agents. Examples of useful anti-acne actives include resorcinol, sulfur, salicylic acid and salicylates, alpha-hydroxy acids, nonsteroidal anti-inflammatory agents, retinoic acid, isoretinoic acid and other retinoid compounds, adapalene, tazarotene, azelaic acid and azelaic acid derivatives, antibiotic agents, such as erythromycin and clyndamycin, zinc salts and complexes, and combinations thereof, in a therapeutically effective concentration.

In addition to solid matter, the compositions may further include a safe and effective amount of one or more anti-wrinkle actives or anti-atrophy actives, which can be easily delivered by spreading a foam onto the skin. Exemplary anti-wrinkle/anti-atrophy active agents suitable for use in the compositions of the present invention include sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate).

Ingredients that are known in the art of pharmacology and cosmetology to treat dermatitis, minor skin irritations, sunburn, heat burn, radiation burn, and inhibit inflammation can also be beneficially incorporated in the foam of the present invention. Examples of such active agents include chamomile extract (matricaria recutitia), cucumber distillate (cucumis sativus), lavender water (lavendula angustifolia), rose water (rosa damascena), witch hazel (hamamelis virginiana), allantoin, bisabolol, rosehip oil, calendula oil, azulaene, menthol and camphor.

The composition may be contained in and dispensed from a container capable of withstanding the pressure of the propellant gas and having an appropriate valve/nozzle for dispensing the composition as foam under pressure. A customary liquefied or compressed gas propellant can be added, in the amount of about 5-25% of the total composition. Liquefied propellants are gases that exist as liquids under pressure, including high purity hydrocarbons such as propane, isobutane and n-butane, dimethyl ether and chlorofluorocarbons (CFCs). Compressed gasses are exemplified by air, nitrogen and carbon dioxide.

The composition may be placed on a patch, occlusive tape or the skin-contact compartment of a transdermal delivery apparatus and applying such object onto the skin, in order to attain effective superficial treatment or enhanced penetration of the drug into the skin or through the skin. Utilizing such strategy, one can apply drugs, which are currently administered systemically or that require transdermal delivery, in the preferred therapeutic system of the present invention. Examples for such drugs are nicotine, testosterone and other male hormones and male hormone precursors, estrogen and other female hormones and hormone precursors, growth hormone, insulin, caffeine, steroidal and non-steroidal antiinflammatory agents and thyroid hormone substitutes.

Thus, by including appropriate therapeutically-active solid particles and optional active agents, the foamable composition are useful in treating a patient having any one of a variety of dermatological disorders (also termed “dermatoses”), such as classified in a non-limiting exemplary manner according to the following groups:
Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash;
Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma;
Fungal Infections including dermatophyte infections, yeast infections; parasitic infections including scabies, pediculosis, creeping eruption;
Viral infections;
Disorders of hair follicles and sebaceous glands including acne, rosacea, perioral dermatitis, hypertrichosis (hirsutism), alopecia, including male pattern baldness, alopecia areata, alopecia universalis and alopecia totalis; pseudofolliculitis barbae, keratinous cyst;
Scaling papular diseases including psoriasis, pityriasis rosea, lichen planus, pityriasis rubra pilaris;
Benign tumors including moles, dysplastic nevi, skin tags, lipomas, angiomas, pyogenic granuloma, seborrheic keratoses, dermatofibroma, keratoacanthoma, keloid;
Malignant tumors including basal cell carcinoma, squamous cell carcinoma, malignant melanoma, paget's disease of the nipples, kaposi's sarcoma;
Reactions to sunlight including sunburn, chronic effects of sunlight, photosensitivity;
Bullous diseases including pemphigus, bullous bemphigoid, dermatitis herpetiformis, linear immunoglobulin A disease;
Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;
Disorders of comification including Ichthyosis, keratosis Pilaris, calluses and corns, actinic keratosis;
Pressure sores;
Disorders of sweating; and
Inflammatory reactions including drug eruptions, toxic epidermal necrolysis; erythema multiforme, erythema nodosum, granuloma annulare.

The same advantage is expected when the composition is topically applied to body cavities, mucosal membranes, the oral cavity, the nasal cavity, the ear canal, the eye, the vagina the gastrointestinal tract and the rectum.

It is useful to treat conditions such as bacterial infection, fungal infection, yeast infection, viral infection, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, cnal abscess/fistula, anal cancer, anal fissure, anal warts, Crohn's disease, hemorrhoids, anal itch, pruritus ani, fecal incontinence, constipation, polyps of the colon and rectum.

The pharmaceutical carrier according to the present invention can also be used to prepare cosmetics for beauty purpose by adding into skin care agents and perfume. The invention is described with reference to the following examples. This invention is not limited to these examples and experiments. Many variations will suggest themselves and are within the full intended scope of the appended claims.

### Example 1

### General Procedure for Preparing Foamable Composition

The general process, as typically exemplified in Example 1 may be applied in order to produce the composition of the present invention.

Aqueous Phase: Water gelling agent and surface-active agent are dispersed and dissolved in water, with agitation. The solution is warmed to 50-700C. Water soluble cosmetic or pharmaceutical active ingredients and optional water soluble ingredients are added with agitation to the Aqueous Phase mixture.

Hydrophobic Phase: The hydrophobic solvent is heated to same temperature. Foam adjuvant agent is added to preheated hydrophobic solvent. Oil soluble cosmetic or pharmaceutical active ingredients* and optional oil soluble formulation ingredients are added with agitation to the Hydrophobic Phase mixture.

Levigation: the solid particulate matter is added to and thoroughly mixed and dispersed into portion of the non-solvent hydrophobic or aqueous phase until homogeneous mixture is obtained. Laboratory scale levigation is performed with mortar and pestle and large scale is performed with mechanical colloidal mill or homogenizing mixer or ball mill drum. The levigation process serves to control particle size and ensure uniform incorporation of the particulate matter into the whole body of the formulation.

The warm Hydrophobic Phase is gradually poured into the warm Aqueous Phase, with agitation, followed by Ultraturax homogenization. The mixture is allowed to cool down to ambient temperature. In case of heat sensitive active ingredients, the active ingredient is added with agitation to the mixture after cooling to ambient temperature. The mixture, at ambient temperature, is added to an aerosol container, the container is sealed and appropriate amount of propellant (5-25 w % of the composition mass) is added under pressure into the container.

### Example 2

### Exemplary Foam Formulations With Solid Matter

| | 1 | 2 | 3 | 4 | 5 | 6 |
|---|---|---|---|---|---|---|
| Ingredient | % w/w | % w/w | % w/w | % w/w | % w/w | % w/w |
| Mineral oil | 12.00 | 12.00 | 12.00 | 12.00 | 12.00 | 10.00 |
| Isopropyl myristate | 12.00 | 12.00 | 12.00 | 12.00 | 12.00 | 10.00 |
| Dimeticone V100 | 3.00 | 3.00 | 3.00 | 3.00 | 3.00 | |
| Glyceryl monostearate | 0.50 | 0.50 | 0.50 | 0.50 | 0.50 | 0.50 |
| Zinc oxide | 10.00 | 15.00 | 15.00 | 20.00 | 25.00 | |
| Titanium Dioxide | | | | | | 20.00 |
| Alpha-Bisabolol | 0.20 | 0.20 | 0.20 | 0.20 | 0.20 | |
| MYRJ 52 | 3.00 | 3.00 | 3.00 | 3.00 | 3.00 | 3.00 |
| Avicel CL611 (microcrystalline | 2.00 | 1.00 | 2.00 | 2.00 | 2.00 | 2.00 |
| cellulose + carboxymethyl | | | | | | |
| cellulose) | | | | | | |
| TWEEN 80 | 1.00 | 1.00 | 1.00 | 1.00 | 1.00 | 1.00 |
| Cocoamidopropylbethaine | 0.50 | 0.50 | 0.50 | 0.50 | 0.50 | 0.50 |
| D-Panthenol 50P | 10.00 | 10.00 | 10.00 | 10.00 | 10.00 | |
| Preservative | 0.30 | 0.30 | 0.30 | 0.30 | 0.30 | 0.30 |
| Purified water | qs | qs | qs | qs | qs | qs |
| | 100.0 | 100.0 | 100.0 | 100.0 | 100.0 | 100.0 |
| Centrifugation test | | | | | | |
| 10000/3 min | stable | stable | stable | stable | stable | stable |
| 10000/10 min | stable | stable | Presipi- | stable | stable | stable |
| | | | tate | | | |
| Foam Quality | E | E | E | E | E | E |
| Density | 0.04 | 0.03 | 0.03 | 0.03 | 0.03 | 0.04 |

### Example 3

### Comparison Between Microcrystalline Cellulose and Polyvinyl Pirrolidone (PVP) as a Stabilizing/gelling Agent

The following table compares foams including microcrystalline cellulose, vs. The corresponding foam with PVP and stabilizing/gelling agent. It clearly shows that microcrystalline cellulose is superior to PVP in forming a stable foam, which does not allow solid matter sedimentation and has excellent texture and low density.

| | PVP Foam | Cellulose Foam |
|---|---|---|
| | % w/w | % w/w |
| Ingredient | | |
| Mineral oil | 30.00 | 30.00 |
| Dimeticone V100 | 3.00 | 3.00 |
| Zinc oxide | 10.00 | 10.00 |
| Arlacel 135 | 2.00 | 2.00 |
| PVP K90 | 2.00 | — |
| Avicel CL611 | — | 2.00 |
| (micronized cellulose + CMC) | | |
| TWEEN 80 | 2.00 | 2.00 |
| Cocoamidopropylbethaine | 1.00 | 1.00 |
| D-Panthenol 50P | 10.00 | 10.00 |
| Benzalconium chlorid | 0.20 | 0.20 |
| Water pur. | qs 100.0 | qs 100.0 |
| Centrifugation Test | | |
| 10000/3 min | Sedimentation | stable |
| 10000/10 min | Sedimentation | stable |
| FoamQuality | FG | E |
| Density | N/A | 0.05 |

### Example 4

### Sunblock Foam

| | Low SPF Foam | High SPF Foam |
|---|---|---|
| | % w/w | % w/w |
| Ingredient | | |
| Mineral oil | 12.50 | 12.50 |
| Micronized titanium dioxide | 5.00 | 10.00 |
| P-aminobenzoic acid | | 3.00 |
| Oxybenzone | | 3.00 |
| Menthyl anthranilate | | 3.00 |
| Arlacel 135 | 2.00 | 2.00 |
| Avicel CL611 | 2.00 | 2.00 |
| (micronized cellulose + CMC) | | |
| TWEEN 80 | 2.00 | 2.00 |
| Cocoamidopropylbethaine | 1.00 | 1.00 |
| D-Panthenol 50P | 10.00 | 10.00 |
| Benzalconium chlorid | 0.20 | 0.20 |
| Water pur. | qs 100.0 | qs 100.0 |
| Centrifugation Test | | |
| 10000/3 min | stable | stable |
| 10000/10 min | stable | slight |
| | | sedimentation |
| FoamQuality | E | E |
| Density | 0.04 | 0.04 |

### Example 5

### Anti-acne Foam

| | Formula A1 | Formula A2 |
|---|---|---|
| | % w/w | % w/w |
| Ingredient | | |
| Mineral oil | 12.50 | 12.50 |
| Benzoyl peroxide | 10.00 | 10.00 |
| Clindamycin | | 1.00 |
| Arlacel 135 | 2.00 | 2.00 |
| Avicel CL611 | 2.00 | 2.00 |
| (micronized cellulose + CMC) | | |
| TWEEN 80 | 2.00 | 2.00 |
| Cocoamidopropylbethaine | 1.00 | 1.00 |
| D-Panthenol 50P | 10.00 | 10.00 |
| Benzalconium chlorid | 0.20 | 0.20 |
| Water pur. | qs 100.0 | qs 100.0 |
| Centrifugation Test | | |
| 10000/3 min | stable | stable |
| 10000/10 min | stable | slight |
| | | sedimentation |
| FoamQuality | E | E |
| Density | 0.04 | 0.04 |

### Example 6

### Wound Healing Foam

| | Formula W1 | Formula W2 |
|---|---|---|
| % w/w | % w/w | |
| Ingredient | | |
| Mineral oil | 12.50 | 12.50 |
| Colloidal silver | 2.00 | 2.00 |
| Lidocaine | | 4.00 |
| Arlacel 135 | 2.00 | 2.00 |
| Avicel CL611 | 2.00 | 2.00 |
| (micronized cellulose + CMC) | | |
| TWEEN 80 | 2.00 | 2.00 |
| Cocoamidopropylbethaine | 1.00 | 1.00 |
| D-Panthenol 50P | 10.00 | 10.00 |
| Benzalconium chlorid | 0.20 | 0.20 |
| Water pur. | qs 100.0 | qs 100.0 |
| Centrifugation Test | | |
| 10000/3 min | stable | stable |
| 10000/10 min | stable | slight |
| | | sedimentation |
| FoamQuality | E | E |
| Density | 0.04 | 0.04 |

1. A foamable composition comprising:
about 2 to about 30% by weight solid particles;
about 2 to about 75% by weight hydrophobic solvent;
about 10 to about 85% by weight water;
about 0.1% to about 5% by weight surface-active agent;
about 0.1% to about 5 wt % by weight stabilizer/gelling agent; and
a liquefied or compressed gas propellant in a container, which upon release provides a breakable foam suitable for topical administration.

2. The foamable composition of claim 1, wherein the hydrophobic solvent concentration is about 5-10% by weight of composition.

3. The foamable composition of claim 1, wherein the hydrophobic solvent concentration is about 10-20% by weight of composition.

4. The foamable composition of claim 1, wherein the hydrophobic solvent concentration is about 20-75% by weight of composition.

5. The foamable composition of claim 1, wherein the composition has viscosity before foaming of between about 100 CPS and about 10,000 CPS.

6. The foamable composition of claim 1, wherein the composition has a viscosity before foaming of between about 500 CPS and about 8,000 CPS.

7. The foamable composition of claim 1, wherein the composition has a viscosity before foaming of between about 1000 CPS and about 5,000 CPS.

8. The foamable composition of claim 1, further comprising about 0.1% to about 5% foam adjuvant agent.

9. The foamable composition of claim 1, wherein the solid particles are an agent that is not soluble in the foamable composition more than 10% of the concentration intended to be included in the foamable composition.

10. The foamable composition of claim 1, wherein the solid particles are selected from the group consisting of metallic oxides, silicon containing solid matter, carbon, oxidizing agents, pigments, cosmetic scrub materials, metal particles and metallic silver.

11. The foamable composition of claim 10, wherein the metallic oxide is selected from the group consisting of titanium dioxide, zinc oxide, zirconium oxide, and iron oxide and mixtures thereof.

12. The foamable composition of claim 11, wherein titanium dioxide has an average primary particle size of from about 15 nm to about 100 nm.

13. The foamable composition of claim 11, wherein zinc oxide has an average primary particle size of from about 15 nm to about 150 nm.

14. The foamable composition of claim 11, wherein zirconium oxide has an average primary particle size of from about 15 nm to about 150 nm.

15. The foamable composition of claim 11, wherein iron oxide has an average primary particle size of from about 15 nm to about 500 nm.

16. The foamable composition of claim 1, wherein the solid particles are micronized to form particles having primary size of less than 15 nm.

17. The foamable composition of claim 11, wherein the solid particles comprise an inorganic sunscreen, present in the amount of from about 0.1% to about 20% by weight.

18. The foamable composition of claim 17, wherein the inorganic sunscreen is present in the amount of from about 0.5% to about 10% by weight.

19. The foamable composition of claim 17, wherein the inorganic sunscreen is present in the amount of from about 1% to about 5% by weight.

20. The foamable composition of claim 10, wherein the silicon containing solid matter is selected from the group consisting of silicone oxide and talc.

21. The foamable composition of claim 10, wherein the carbon comprises amorphous carbon or graphite.

22. The foamable composition of claim 10, wherein the oxidizing agents are selected from the group consisting of benzoyl peroxide, calcium and magnesium hypochlorite.

23. The foamable composition of claim 10, wherein the cosmetic scrub materials are selected from the group consisting of meals of strawberry seeds, raspberry seeds, apricot seeds, sweet almond, and cranberry seeds.

24. The foamable composition of claim 1, further comprising at least one additional therapeutic agent, selected from the group consisting of an anti-infective, an antibiotic, an antibacterial agent, an antifungal agent, an antiviral agent, an antiparasitic agent, an antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an analgesic, an antiallergic agent, a corticosteroid, a non-steroidal anti-inflammatory drug, an alpha hydroxyl acid, a beta-hydroxy acid, a protein, a peptide, a neuropeptide, a allergen, an immunogenic substance, a haptene, an oxidizing agent, an antioxidant, a retinoid, an antiproliferative agent, an anticancer agent, a photodynamic therapy agent, an anti-wrinkle agent, a radical scavenger, a self-tanning agent, a skin whitening agent, a skin protective agent, an anti-cellulite agent, a massaging oil and an anti-wart agent, a refatting agent, a lubricating agent and mixtures thereof.

25. The foamable composition of claim 1, wherein the foamable composition is thixotropic.

26. The foamable composition of claim 1, wherein the gelling/stabilizing agent is present in an amount in the range of 0.1% to about 5 wt % by weight of the foamable composition.

27. The foamable composition of claim 1, wherein the gelling/stabilizing agent is present in an amount in the range of about 0.5% to about 3 wt % by weight of the foamable composition.

28. The foamable composition of claim 1, wherein the gelling/stabilizing agent is present in an amount in the range of about 1% to about 2 wt % by weight of the foamable composition.

29. The foamable composition of claim 1, wherein the gelling/stabilizing agent comprises hydrocolloid selected from natural cellulose gums and derivatives, polysaccharides and derivatives, microcrystalline cellulose, sodium carboxy methyl cellulose, fumed silica, bentonite, Xanthan gum, carrageenan, polyacrylate and mixtures thereof.

30. The foamable composition of claim 1, wherein the surface-active agent consists essentially of one or more non-ionic surfactants.

31. The foamable composition of claim 1, wherein the surface-active agent is a mixture of a non-ionic surfactant and an ionic surfactant in a 100:1 to 6:1 ratio.

32. The foamable composition of claim 1 or 6, wherein the combined amount of foam adjuvant agent, surface active agent and water gelling agent is less than about 8% (w/w).

33. The foamable composition of claim 32, wherein the combined amount of foam adjuvant agent, surface active agent and water gelling agent is less than about 5% (w/w).

34. The foamable composition of claim 1, wherein the solid particles are selected for the treatment of a superficial condition.

35. The foamable composition of claim 1, wherein the solid particles are selected for the treatment of a disorder of the skin, body cavities, mucosal membranes, the oral cavity, the nasal cavity, the eye, the ear canal, the vagina, the gastrointestinal tract and the rectum .

36. The foamable composition of claim 1, wherein the solid particles are selected for the treatment of a disorder selected from the group of bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and
wherein said disorder is known to be responsive to treatment with said therapeutic solid particles.

37. The foamable composition of claim 1, wherein the solid particles are selected for the treatment of wounds, burns, cuts and ulcers.

38. The foamable composition of claim 1, wherein the solid particles are a sun-block agent.

39. The foamable composition of claim 38, further comprising a soluble sunscreen agent.

40. The foamable composition of claim 38, further comprising a skin whitening agent.

41. A foamable composition for the treatment of diaper dermatitis comprising:
about 6% to about 20% of a metal oxide selected from the group consisting of zinc oxide, zirconium oxide, iron oxide, titanium dioxide and mixtures thereof;
about 10% to about 40% hydrophobic solvent;
about 40% to about 80% water;
about 0.1% to about 5% surface-active agent; and
about 0.5% to about 5% stabilizer/gelling agent.

42. The foamable composition of claim 41, wherein the stabilizer/gelling agent is in an amount from about 1% to about 2% by weight.

43. The foamable composition of claim 41, further comprising:
about 0.1% to about 5% foam adjuvant agent.

44. The foamable composition of claim 41, whereby upon discharge from the aerosol can, the foamable composition forms a mass having density between 0.01 gr/mL and 0.1 gr/mL.

45. The foamable composition of claim 41, further comprising an agent selected from the group of local anesthetic agents, anti-inflammatory agents, corticosteroids, antifungal agents antibacterial agents and antiviral agents.

46. A method of treating, alleviating or preventing a disorder, comprising:
administering topically to a subject having said disorder a therapeutically effective amount of a breakable foam composition comprising:
(a) a foamable composition comprising:
about 2 to about 30% solid particles;
about 2 to about 75% hydrophobic solvent;
about 10 to about 85% water;
about 0.1% to about 5% surface-active agent; and
about 0.1% to about 5 wt % by weight stabilizer/gelling agent; and
(b) a liquefied or compressed gas propellant in a container,
which upon release provides a breakable foam suitable for topical administration.

47. The method of claim 46, wherein the disorder from the group consisting of body cavity disorders, mucosal membrane disorders, oral cavity disorders, nasal cavity disorders, ear canal disorders, eye disorders and disorders of the vagina and the rectum.

48. The method of claim 46, wherein the disorder is selected from the group consisting of bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

49. The method of claim 46, wherein the target site of treatment is selected from the group consisting of body cavities, mucosal membranes, the oral cavity, the nasal cavity, the ear canal, the eye, the vagina, the gastrointestinal system and the rectum.

50. The method of claim 46, wherein the hydrophobic solvent concentration is about 5-10% by weight of composition.

51. The method of claim 46, wherein the hydrophobic solvent concentration is about 10-20% by weight of composition.

52. The method of claim 46, wherein the hydrophobic solvent concentration is about 20-75% by weight of composition.

53. The method of claim 46, wherein the composition has viscosity before foaming of between about 100 CPS and about 10,000 CPS.

54. The method of claim 46, wherein the composition has a viscosity before foaming of between about 500 CPS and about 8,000 CPS.

55. The method of claim 46, wherein the composition has a viscosity before foaming of between about 1000 CPS and about 5,000 CPS.

56. The method of claim 46, further comprising a foamable composition comprising about 0.1% to about 5% foam adjuvant agent.
